(19) United States
(12) Patent Application Publication
(10) Pub. No.: US 2026/0294533 A1
(43) Pub. Date: Oct. 1, 2026

(54) AUTOMATIC CALIBRATION FOR DISTANCE MEASUREMENT DURING LASER LITHOTRIPSY PROCEDURE

(71) Applicant: Lumenis Ltd., Yokneam Ilit (IL)

(72) Inventors: Amir Finkel, Zichron Yaakov (IL); Saman Naser, Tira (IL); Asya Mikhaylov, Kiryat Gat (IL)

(73) Assignee: Lumenis Ltd., Yokneam Ilit (IL)

(21) Appl. No.: 19/634,351

(22) Filed: Mar. 31, 2026

Related U.S. Application Data
(60) Provisional application No. 63/781,560, filed on Apr. 1, 2025.

(51) Int. Cl. A61B 18/20; G16H 20/40; A61B 17/00; A61B 18/00

(52) U.S. Cl. CPC A61B 18/20; G16H 20/40; A61B 2017/00066; A61B 2018/00642; A61B 2018/2025

(57) ABSTRACT

The present disclosure provides a surgical laser system configured to measure a distance between a distal end of an optical fiber in which laser energy is transmitted and a target where reflections from the distal end of the optical fiber are accounted for in measuring the distance.

### CROSS-REFERENCE TO RELATED APPLICATIONS

This application claims the benefit of priority under 35 USC § 119 to United States Provisional Patent Application Serial No. 63/781,560, filed Apr. 1, 2025, which is incorporated by reference herein in its entirety and for all purposes.

### TECHNICAL FIELD

The present disclosure generally relates to surgical laser systems used in medical or therapeutic procedures. Particularly, but not exclusively, the present disclosure relates to automatically calibrating a distance measurement system for measuring a distance between a distal end of an optical fiber and a target.

### BACKGROUND

Introduction of lasers into the medical field and the development of fiber optic technologies that use lasers has opened numerous applications in treatments, diagnostics, therapies, and the like. Such applications range from invasive and non-invasive treatments to endoscopic surgeries and image diagnostics. For instance, in urinary stone treatment, some stones are required to be fragmented into smaller pieces. A technology known as laser lithotripsy may be used for such fragmenting processes.

In an example lithotripsy procedure, a rigid or flexible ureteroscope is placed through the urinary tract for illumination and imaging. Simultaneously, an optical fiber is inserted through a working channel of the ureteroscope, to a target location (e.g., to the location where the stone is present in the bladder, ureter, or kidney). The laser is then activated to fragment the stone into smaller pieces or to dust it.

These treatments require high amounts of accuracy to ensure that the laser is aimed at the right target (stone, tissue, etc.), to achieve the clinical objectives (e.g., stone fragmentation, stone dusting, tissue coagulation, etc.). Further, the procedure efficiency and/or efficacy can be optimized where the distance between the target and the distal end of the optical fiber (e.g., where the laser radiation is emitted) is known.

Thus, there is a need for improved surgical laser systems configured to measure, or estimate, the distance between the target and the distal end of the optical fiber.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

The present disclosure applies to surgical laser systems with light detectors arranged to detect optical signals and provides to measure light reflected from the distal end of the optical fiber and to calibrate measurement of the distance between the distal end of the optical fiber and the target based on the light reflected from the distal end of the optical fiber.

Some embodiments of the disclosure can be implemented as a method for a surgical laser system. The method can comprise generating, via a laser source, an interrogation laser beam; illuminating, via an optical fiber coupled to the surgical laser system, a target with the interrogation laser beam; measuring, via at least one signal detector, an intensity of a reflected light beam, wherein the reflected light beam comprises target reflections and parasitic reflections; deriving a corrected intensity based on the measured intensity and one or more parasitic intensities; determining whether the corrected intensity is less than a threshold value; and updating at least one of the parasitic intensities based on the corrected intensity responsive to a determination that the corrected intensity is less than the threshold value.

In further embodiments, the method can comprise deriving a normalized intensity based on the measured intensity and a dark current intensity, wherein deriving the corrected intensity is based on the normalized intensity and the one or more parasitic intensities.

In further embodiments, the method can comprise applying a smoothing filter to the measured intensity prior to deriving the normalized intensity.

In further embodiments, the method can comprise measuring, via a reference detector, an intensity of the interrogation laser beam, wherein deriving the normalized intensity based on the measured intensity and the dark current intensity comprises deriving the normalized intensity based on the measured intensity of the reflected light beam, the measured intensity of the interrogation laser beam, and the dark current intensity.

In further embodiments, the method can comprise determining whether a standard deviation of the normalized intensity value for N last measured intensities is less than a stability threshold value; and updating the at least one of the parasitic intensities based on the corrected intensity responsive to the determination that the corrected intensity is less than the threshold value and a determination that the standard deviation of the normalized intensity value for the N last measured intensities is less than the stability threshold value.

In further embodiments, the method can comprise measuring, via the at least one signal detector, the dark current intensity during a period when the interrogation laser beam is off.

In further embodiments of the method, the one or more parasitic intensities comprise a distal facet reflection value and an internal reflection value.

In further embodiments of the method, deriving the corrected intensity based on the measured intensity and the one or more parasitic intensities comprises subtracting the distal facet reflection value and the internal reflection value from the measured intensity, and wherein the threshold value is substantially zero (0).

In further embodiments of the method, updating at least one of the parasitic intensities based on the corrected intensity comprises setting the distal facet reflection value as the corrected intensity minus the internal reflection value.

In further embodiments of the method, deriving the corrected intensity based on the measured intensity and the one or more parasitic intensities comprises subtracting the internal reflection value from the measured intensity, and wherein the threshold value is equal to the distal facet reflection value.

In further embodiments of the method, updating at least one of the parasitic intensities based on the corrected intensity comprises setting the distal facet reflection value as the corrected intensity.

In further embodiments of the method, the interrogation laser beam comprises a plurality of wavelength components, measuring, via the at least one signal detector, the intensity of the reflected light beam comprises measuring the intensity of the reflected light beam at each of the plurality of wavelength components, the one or more parasitic intensities comprises one or more parasitic intensities at each of the plurality of wavelength components, deriving the corrected intensity based on the measured intensity and the one or more parasitic intensities comprises deriving, for each of the plurality of wavelength components, the corrected intensity based on the measured intensity and the one or more parasitic intensities at the respective wavelength component, the threshold value comprises a threshold value at each of the plurality of wavelength components, determining whether the corrected intensity is less than the threshold value comprises determining, for each of the measured intensities, whether the measured intensity is less than the threshold value at the respective wavelength component, and updating at least one of the parasitic intensities based on the corrected intensity responsive to a determination that the corrected intensity is less than the threshold value comprises updating, for each of the wavelength components, at least one of the parasitic intensities at the respective wavelength component.

In further embodiments, the method can comprise estimating a distance between a distal end of the optical fiber and the target based on the corrected intensity responsive to a determination that the corrected intensity is not less than the threshold value.

In further embodiments of the method, the first wavelength component is 1310 nanometers (nm), wherein the second wavelength component is 1340 nm, and wherein the third wavelength component is 1431 nm.

Some embodiments of the disclosure can be implemented as a surgical laser system. The surgical laser system can comprise at least one laser source configured to output an interrogation laser beam; at least one signal detector configured to measure an intensity of a reflected light beam; a processor; and a memory comprising instructions, which when executed by the processor cause the processor to send a control signal to the at least one laser source to cause the at least one laser source to generate the interrogation laser beam, receive electrical signals from the at least one signal detector, the electrical signals comprising indications of an intensity of a reflected light beam, wherein the reflected light beam comprises target reflections and parasitic reflections, and wherein the reflected light beam is responsive to incidence of the interrogation laser beam on a target, derive a corrected intensity based on the intensity and one or more parasitic intensities, determine whether the corrected intensity is less than a threshold value, and update at least one of the parasitic intensities based on the corrected intensity responsive to a determination that the corrected intensity is less than the threshold value.

In further embodiments of the surgical laser system, the instructions when executed by the processor further cause the processor to derive the normalized intensity based on the measured intensity and a dark current intensity and to derive the corrected intensity is based on the normalized intensity and the one or more parasitic intensities.

In further embodiments of the surgical laser system, the instructions when executed by the processor further cause the processor to apply a smoothing filter to the measured intensity prior to deriving the normalized intensity.

In further embodiments of the surgical laser system, the instructions when executed by the processor further cause the processor to receive electrical signals from a reference detector, the electrical signals comprising indications of an intensity of the interrogation laser beam and to derive the normalized intensity based on the intensity of the reflected light beam, the intensity of the interrogation laser beam, and the dark current intensity.

In further embodiments of the surgical laser system, the instructions when executed by the processor further cause the processor to determine whether a standard deviation of the normalized intensity value for N last measured intensities is less than a stability threshold value; and update the at least one of the parasitic intensities based on the corrected intensity responsive to the determination that the corrected intensity is less than the threshold value and a determination that the standard deviation of the normalized intensity value for the N last measured intensities is less than the stability threshold value.

In further embodiments of the surgical laser system, the instructions when executed by the processor further cause the processor to receive electrical signals from the at least one signal detector during a period when the interrogation laser beam is off, the electrical signals comprising indications of the dark current intensity.

In further embodiments of the surgical laser system, the one or more parasitic intensities comprise a distal facet reflection value and an internal reflection value.

In further embodiments of the surgical laser system, the instructions when executed by the processor further cause the processor to subtract the distal facet reflection value and the internal reflection value from the measured intensity to derive the corrected intensity when the threshold value is substantially zero (0).

In further embodiments of the surgical laser system, the instructions when executed by the processor further cause the processor to set the distal facet reflection value as the corrected intensity minus the internal reflection value to update at least one of the parasitic intensities based on the corrected intensity.

In further embodiments of the surgical laser system, the instructions when executed by the processor further cause the processor to subtract the internal reflection value from the measured intensity to derive the corrected intensity when the threshold value is equal to the distal facet reflection value.

In further embodiments of the surgical laser system, the instructions when executed by the processor further cause the processor to set the distal facet reflection value as the corrected intensity to update at least one of the parasitic intensities based on the corrected intensity.

In further embodiments of the surgical laser system, the interrogation laser beam comprises a plurality of wavelength components, wherein the electrical signals received from the at least one signal detector comprises indications of the intensity of the reflected light beam at each of the plurality of wavelength components, wherein the one or more parasitic intensities comprises one or more parasitic intensities at each of the plurality of wavelength components, wherein the corrected intensity comprises a corrected intensity at each of the plurality of wavelength components, wherein the threshold value comprises a threshold value at each of the plurality of wavelength components, and wherein the instructions when executed by the processor further cause the processor to determine whether the corrected intensity is less than the threshold value comprises determining, for each of the plurality of wavelength components, whether the corrected intensity is less than the respective threshold value, and updating, for each of the wavelength components, at least one of the parasitic intensities based on the corrected intensity responsive to a determination that the corrected intensity is less than the respective threshold value.

In further embodiments of the surgical laser system, the first wavelength component is 1310 nanometers (nm), wherein the second wavelength component is 1340 nm, and wherein the third wavelength component is 1431 nm.

In further embodiments of the surgical laser system, the instructions when executed by the processor further cause the processor to estimate a distance between a distal end of the optical fiber and the target based on the corrected intensity responsive to a determination that the corrected intensity is not less than the threshold value.

Some embodiments of the disclosure can be implemented as at least one machine readable storage device comprising instructions that when executed by a processor of a surgical laser system cause the surgical system to generate, via a laser source, an interrogation laser beam; illuminate, via an optical fiber coupled to the surgical laser system, a target with the interrogation laser beam; measure, via at least one signal detector, an intensity of a reflected light beam, wherein the reflected light beam comprises target reflections and parasitic reflections; derive a corrected intensity based on the measured intensity and one or more parasitic intensities; determine whether the corrected intensity is less than a threshold value; and update at least one of the parasitic intensities based on the corrected intensity responsive to a determination that the corrected intensity is less than the threshold value.

In further embodiments of the at least one machine readable storage device, the instructions when executed by the processor further cause the surgical laser system to measure, via the at least one signal detector, a dark current intensity during a period when the interrogation laser beam is off; and derive a normalized intensity based on the measured intensity and the dark current intensity, wherein deriving the corrected intensity is based on the normalized intensity and the one or more parasitic intensities.

In further embodiments of the at least one machine readable storage device, the interrogation laser beam comprises a plurality of wavelength components, measuring, via the at least one signal detector, the intensity of the reflected light beam comprises measuring the intensity of the reflected light beam at each of the plurality of wavelength components, the one or more parasitic intensities comprises one or more parasitic intensities at each of the plurality of wavelength components, deriving the corrected intensity based on the measured intensity and the one or more parasitic intensities comprises deriving, for each of the plurality of wavelength components, the corrected intensity based on the measured intensity and the one or more parasitic intensities at the respective wavelength component, the threshold value comprises a threshold value at each of the plurality of wavelength components, determining whether the corrected intensity is less than the threshold value comprises determining, for each of the measured intensities, whether the measured intensity is less than the threshold value at the respective wavelength component, and updating at least one of the parasitic intensities based on the corrected intensity responsive to a determination that the corrected intensity is less than the threshold value comprises updating, for each of the wavelength components, at least one of the parasitic intensities at the respective wavelength component.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1A illustrates a surgical laser system in accordance with embodiments of the disclosure.

FIG. 1B illustrates a portion of the surgical laser system of FIG. 1A in greater detail.

FIG. 2 illustrates a first interrogation sub-system in accordance with embodiments of the disclosure.

FIG. 3 illustrates a method for controlling a laser source to stabilize signals at a detector in accordance with embodiments of the disclosure.

FIG. 4 illustrates a method for controlling a laser source to stabilize signals at a detector in accordance with embodiments of the disclosure.

FIG. 5 illustrates an aspect of the subject matter in accordance with one embodiment.

FIG. 6 illustrates a computer-readable storage medium in accordance with embodiment of the disclosure.

FIG. 7 illustrates a specially programmed computing system in accordance with embodiments of the disclosure.

### DETAILED DESCRIPTION

The present disclosure applies to surgical laser systems with light detectors arranged to detect optical signals. An illustrative surgical laser system can be provided with both therapeutic and interrogative laser sources and can be configured to measure the distance between a target and a distal end of an optical fiber using the interrogative laser sources. It is to be appreciated that the primary signal for measuring this distance is light that is reflected from the target. However, this signal is mixed with other parasitic reflections (e.g., reflections from internal optical components, reflections from the proximal end of the optical fiber, and reflections from the distal end of the optical fiber). The present disclosure provides systems and methods to measure and account for these parasitic signals as part of an overall distal tip to target measurement process.

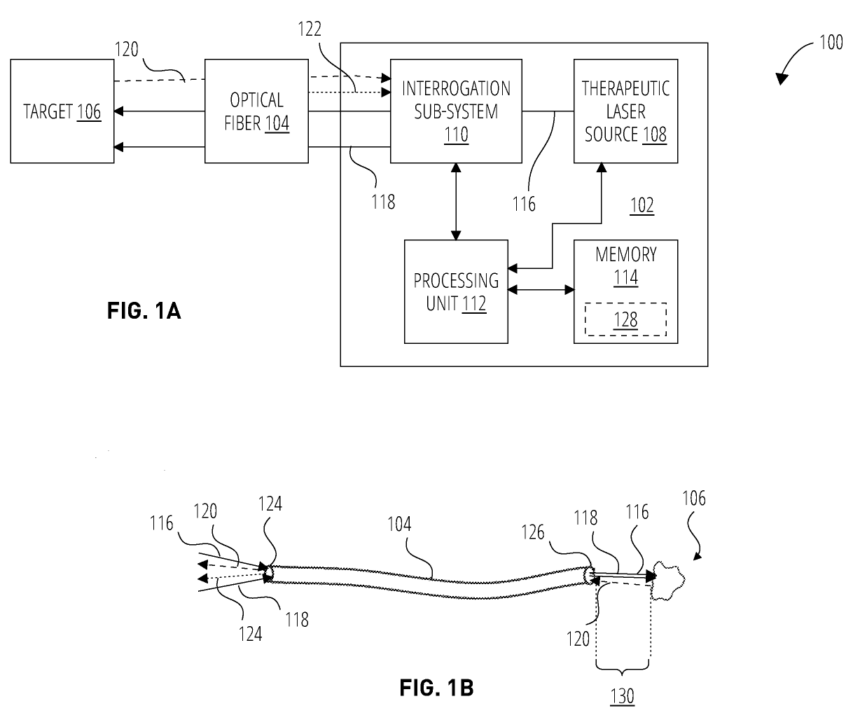

FIG. 1A show an exemplary surgical laser system 100 for estimating a distance between a distal end of an optical fiber and a target, in accordance with some embodiments of the present disclosure. The surgical laser system 100 comprises a surgical laser console 102 and an optical fiber 104. To aid the discussion, the surgical laser system 100 is depicted treating a target 106. In an exemplary lithotripsy procedure, the target 106 can be a stone. However, it will be appreciated that the surgical laser console 102 and optical fiber 104 could be used to treat other types of targets (e.g., tissue, a tumor, a cyst, or the like). With illustrative examples, the target can be disposed in a liquid environment within a subject (e.g., a human, an animal, or the like).

The surgical laser console 102 includes a therapeutic laser source 108, an interrogation sub-system 110, a processing unit 112, and a memory 114. During operation, the therapeutic laser source 108 is configured to generate therapeutic laser pulses 116 while interrogation sub-system 110 is configured to generate interrogation laser pulses 118.

In general, the therapeutic laser source 108 can be any of a variety of laser light sources (e.g., solid-state lasers, gas lasers, diode lasers, and fiber lasers). As a specific example, the therapeutic laser source 108 can be a Holmium (Ho) laser, such as a Ho:YAG laser. In yet another example, the therapeutic laser source 108 can be a Thulium (Tm) fiber laser.

An example of the interrogation sub-system 110 is provided in greater detail below. However, in general the interrogation sub-system 110 is configured to generate interrogation laser pulses 118, which comprise multiple (e.g., two, three, four, etc.) laser beams each having different wavelengths.

The therapeutic laser pulses 116 and the interrogation laser pulses 118 are transmitted towards the target 106 via the optical fiber 104. Reflected laser pulses 120 are light reflected from the target responsive to the therapeutic laser pulses 116 and/or the interrogation laser pulses 118 being incident on the target as well as light reflected from other features, referred to herein as parasitic reflections 122. This is depicted in greater detail in FIG. 1B, which depicts the optical fiber 104. The optical fiber 104 includes both a proximal end 124 and a distal end 126. The proximal end 124 is the end of the optical fiber 104 coupled to the surgical laser console 102 and through which therapeutic laser pulses 116 and interrogation laser pulses 118 enter while the distal end 126 is the end of the optical fiber 104 through which the therapeutic laser pulses 116 and interrogation laser pulses 118 are emitted. During a procedure, the physician can manipulate the distal end 126 of the optical fiber 104 to “point” the optical fiber 104 at the target 106 and to place the distal end 126 within a desired distance to the target 106.

Further, at outlined above, reflected laser pulses 120 is light reflected off the target in response to illumination by therapeutic laser pulses 116 and/or interrogation laser pulses 118. The reflected laser pulses 120 can be received at the distal end 126 of the optical fiber 104 and transmitted to the surgical laser console 102. Other light reflections contaminate the reflected laser pulses 120. For example, reflections from the distal end 126 of the optical fiber 104, the proximal end 124 of the optical fiber 104, and other internal optical components of the interrogation sub-system 110 (e.g., coupling lenses, etc.). These reflections are collectively referred to herein at parasitic reflections 122.

An example interrogation sub-system 110 is described below. However, the general principle relies on the power of the interrogation laser pulses 118 relative to the power of the reflected laser pulses 120. To that end, interrogation sub-system 110 includes detectors or other sensors (described below) configured to measure the power of interrogation laser pulses 118 and reflected laser pulses 120. As such, it is important to account for the parasitic reflections 122. To that end, the present disclosure provides to measure and/or account for the parasitic reflections 122.

The processing unit 112 can include circuitry arranged to execute instructions 128 stored in memory 114, which instructions when executed cause the processing unit 112 to send control signals to the interrogation sub-system 110 to measure parasitic reflections 122 (or portions of the parasitic reflections 122) and account for the measured parasitic reflections 122 as part of measuring the distance 130 between the target 106 and the distal end 126 of the optical fiber 104. With some examples, the processing unit 112 and memory 114 may be provided by a computing device (e.g., a laptop, a desktop, a mobile phone, a tablet, or the like).

It is to be appreciated that the surgical laser console 102 and particularly the therapeutic laser source 108 and interrogation sub-system 110 can include a variety of optical components besides the laser sources described above. For example, therapeutic laser source 108 and interrogation sub-system 110 can include (e.g., in different combinations as may be the case) light sources, polarizers, beam splitters, beam combiners, light detector, wavelength division multiplexers, collimators, circulators, etc.

Further, although not depicted, the surgical laser console 102 could include a display and/or other input and/or output devices configured to provide interaction with the surgical laser console 102 by a user (e.g., physician, or the like).

It is to be appreciated that the target 106 is often in a liquid environment (e.g., urine, water, blood, etc.) For example, during a lithotripsy procedure, the target 106 may be a stone and may be disposed in an environment that includes a mixture of urine, irrigation fluid, and blood. Accordingly, the distal end 126 of the optical fiber 104 will also be disposed in the liquid environment during the procedure. When the therapeutic laser pulses 116 are emitted from the distal end 126 of the optical fiber 104, some of the liquid may be vaporized and form a bubble or bubbles.

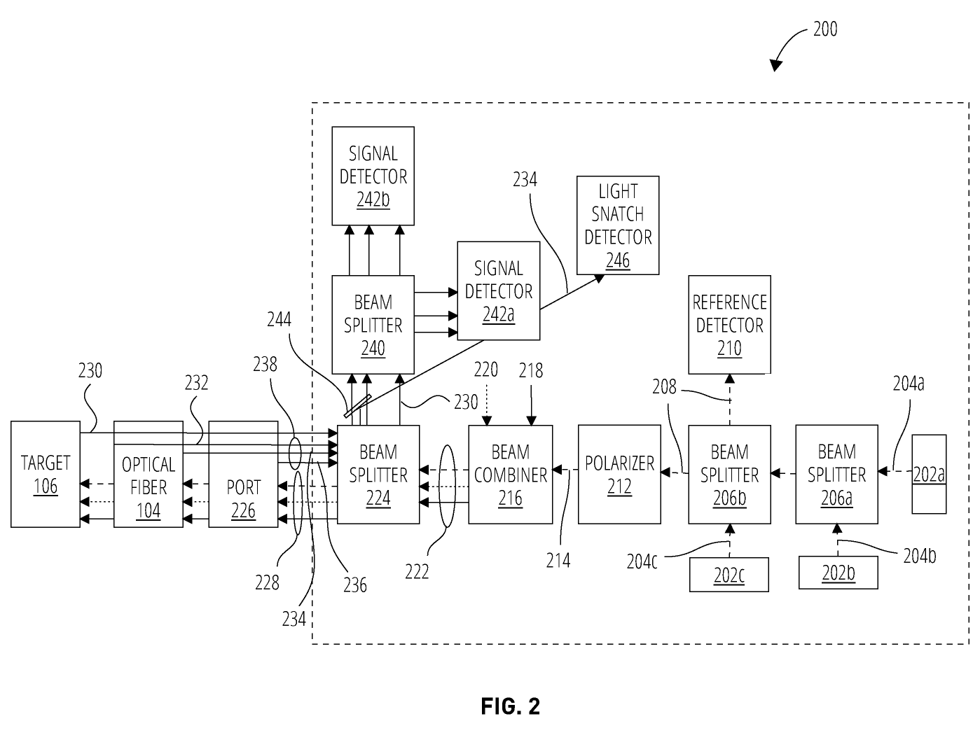

FIG. 2 shows an example interrogation sub-system 200, which can be provided in accordance with some embodiments of the present disclosure. For example, the interrogation sub-system 200 can be provided as the interrogation sub-system 110 of the surgical laser console 102 shown as part of the surgical laser system 100 in FIG. 1A.

As depicted, the interrogation sub-system 200 comprises laser sources 202a, 202b, and 202c, which are configured to generate, respectively, laser beams 204a, 204b, and 204c. The laser sources 202a, 202b, and 202c are each configured to generate laser beams of a different wavelengths. For example, laser source 202a can be configured to generate laser beam 204a having a first wavelength while the laser source 202b can be configured to generate the laser beam 204b having a second wavelength different than the first wavelength. Of note, the first wavelength can have an absorption coefficient (e.g., in water, or the like) higher than an absorption coefficient of the second wavelength. Further, the laser source 202c can be configured to generate the laser beam 204c having yet another wavelength, different from the wavelength of the laser beams 204a and 204b.

As used herein, the terms “high” and “low” are intended to be interpreted relative to each other, or in the alternative relative to a threshold characteristic describing the water absorption of a particular wavelength. For example, a high-water absorption coefficient can be greater than or equal to 150% of the low water absorption coefficient. In a non-limiting example, the laser beam 204a can have a wavelength of approximately 1310 nanometers (nm); laser beam 204b can have a wavelength of approximately 1340 nm; and the laser beam 204c can have a wavelength of approximately 1431 nm. In such an example, the laser beam 204a can be referred to as the “low” absorption beam while the laser beam 204b can be referred to as the “high” absorption beam. Further, the laser beam 204c can be referred to as a calibration laser beam. In other embodiments, the laser beam 204a can have a wavelength of approximately less than or equal to 1310 nanometers (nm); laser beam 204b can have a wavelength of greater than 1310 nm and less than or equal to 1399 nm; and the laser beam 204c can have a wavelength of greater than or equal to 1400 nm.

The laser beams 204a, 204b, and 204c are combined via beam splitters 206a and 206b into interrogation laser beams 208 (e.g., interrogation laser pulses 118 of FIG. 1A, or the like). A reference detector 210 is associated with and in optical communication with the beam splitter 206b. The reference detector 210 is arranged to measure the optical power of each respective component of interrogation laser beams 208 (e.g., component of laser sources 202a, 202b, and 202c).

The beam splitter 206b is further associated with and in optical communication with the polarizer 212. The beam splitter 206b is configured to provide a portion of the interrogation laser beams 208 to the polarizer 212. In some embodiments, the polarity of the polarizer 212 may be pre-configured and arranged to output polarized light beam 214. The interrogation sub-system 200 further includes a beam combiner 216, which is in optical communication with the polarizer 212. In such a manner, the polarized light beam 214 obtained as an output from the polarizer 212 is provided as input to the beam combiner 216. The beam combiner 216 may combine the polarized light beams 214 (e.g., corresponding to the interrogation laser beams 208) with a therapeutic laser beam 218 (e.g., therapeutic laser pulses 116 of FIG. 1A, or the like) and an aiming beam 220, to form combined beam 222.

The interrogation sub-system 200 further includes a beam splitter 224 and a port 226. The beam splitter 224 is arranged in optical communication with the beam combiner 216. As such, the beam splitter 224 can receive the combined beam 222 (comprising the polarized light beam 214, the therapeutic laser beam 218, and the aiming beam 220) from the beam combiner 216 and output the combined beam 222 to the port 226. In such a manner, the beam splitter 224 is optically coupled to the optical fiber 104 via the port 226 such that a portion of the combined beam 222 (e.g., denoted as combined beam portion 228) is transmitted through the optical fiber 104 to be incident on the target 106.

When the combined beam portion 228 is incident on the target 106, the target 106 may reflect some portion of the incident combined beam portion 228 away from the optical fiber 104 and some portion of the light towards the optical fiber 104, wherein the portion of light reflected towards the optical fiber 104 may re-enter the optical fiber 104 (e.g., at the distal end 126 of the optical fiber 104). The portion of the reflected light re-entering the optical fiber 104 at the distal end 126 may be referred to as target reflection beam 230. As noted, the target reflection beams 230 may be contaminated and/or mixed with other reflections. For example, some of the combined beam portion 228 may be reflected from the distal facet (e.g., distal end 126) of the optical fiber 104 as distal facet reflection beam 232 while some of the combined beam portions 228 may be reflected from the proximal facet (e.g., proximal end 124) of the optical fiber 104 as proximal facet reflection beam 234. Further, some of the combined beam portion 228 may be reflected from other internal optical components (e.g., port 226, coupling lenses, etc.) as internal reflection beam 236. The collection of distal facet reflection beams 232, proximal facet reflection beams 234, and internal reflection beam 236 are referred to as parasitic reflected beams 238 (e.g., parasitic reflections 122 of FIG. 1A, or the like).

The present disclosure provides to account for the parasitic reflected beams 238 by either eliminating them or measuring them such that they can be accounted for and considered during measurement of the distance 130 between the distal end 126 of the optical fiber 104 and the target 106.

The parasitic reflected beams 238 may be subjected to the beam splitter 224, which directs the parasitic reflected beams 238 to another beam splitter 240. The beam splitter 240 may be in optical communication with signal detectors 242a and 242b. In some examples, the beam splitter 240 may be a polarizing beam splitter. As such portions of parasitic reflected beams 238 having one polarity (e.g., P) may be transmitted to one of the signal detectors 242a or 242b while portions of the parasitic reflected beams 238 having the other polarity (e.g., S) may be transmitted to the other signal detector 242a or 242b.

The interrogation sub-system 200 further includes a light snatch mirror 244 disposed between beam splitter 224 and the beam splitter 240 and configured to direct the proximal facet reflection beam 234 to a light snatch detector 246, thereby eliminating the proximal facet reflection beam 234 from reaching the signal detectors 242a and 242a and as a result interfering with the measurement. The complete details of eliminating the proximal facet reflection beam 234 from the parasitic reflected beams 238 is beyond the scope of this disclosure. However, the general principle relies on the fact that the proximal facet reflection beams 234 have a different angular rotation than other parasitic beams, and as such, can be selectively directed towards light snatch detector 246 by light snatch mirror 244 without interfering with the other components of parasitic reflected beams 238.

Thus, only the remaining components of parasitic reflected beams 238 (e.g., distal facet reflection beam 232 and internal reflection beam 236) as well as the target reflection beam 230 reach the signal detectors 242a and 242b. The signal detectors 242a and 242b are configured to measure intensities of the components of parasitic reflected beams 238 and the target reflection beam 230 and to transmit the intensities to the processing unit 112. Accordingly, the processing unit 112 may estimate the distance 130 between the distal end 126 of the optical fiber 104 and the target 106 based on the measured intensities.

Complete details of measuring the distance 130 between the distal end 126 of the optical fiber 104 and the target 106 are beyond the scope of this disclosure. However, it is to be appreciated that for accurate measurements (1) the interrogation laser beams 208 and the associated parasitic reflected beams 238 should travel through the liquid environment (e.g., water, or the like) to take advantage of the difference in absorption coefficients between the components of the interrogation laser beams 208; and (2) the parasitic reflected beams 238 associated with the therapeutic laser beam 218 need to either be eliminated or considered.

It is to be appreciated that internal reflection beam 236 are fixed and do not change with time. As such, these signals can be measured once and the measurements used in an overall distance measurement algorithm.

Further it is to be appreciated that the distal facet reflection beams 232 changes during the procedure. For example, the surface of the distal end 126 of the optical fiber 104 is continuously damaged due to the energy impact from the therapeutic laser beam 218 and in instances where fragments of the target 106 impact the surface. Changes to the surface of the distal end 126 of the optical fiber 104 have different, or varying, impacts to each of the different components of the interrogation laser beams 208. Additionally, other phenomenon can contribute to the dynamic nature of the distal facet reflection beam 232, such as, for example, bubbles near the surface of the distal end 126 of the optical fiber 104 forming and collapsing will affect the distal facet reflection beam 232.

Since the distal facet reflection beam 232 changes during the procedure, it cannot be measured upfront like the internal reflection beam 236. Further, the amplitude of the changes in the distal facet reflection beams 232 are often higher than target reflection beams 230. As such, the present disclosure provides to measure the distal facet reflection beam 232 and calibrate and/or adjust the distance measurement algorithm based on the measurement of the distal facet reflection beam 232.

With some embodiments, the processing unit 112 can be configured to execute instructions 128 to measure the distance 130 between the distal end 126 of the optical fiber 104 and the target 106 based on signals received from the reference detector 210 as well as the signal detectors 242a and 242b. Examples of measuring the distance 130 are provided in greater detail below.

However, in general, the disclosure provides to measure distal facet reflection beam 232 during periods where the distance 130 between the distal end 126 of the optical fiber 104 and the target 106 is great enough such that all of the interrogation laser beams 208 is absorbed by the liquid medium, and therefore, target reflection beams 230 is substantially zero.

As outlined above, proximal facet reflection beams 234 is eliminated and internal reflection beam 236 is known and can thus be subtracted from the parasitic reflected beams 238. Accordingly in periods where the distance 130 between the distal end 126 of the optical fiber 104 and the target 106 are great enough that interrogation laser beams 208 is substantially absorbed by the liquid medium, the entire signal reaching the signal detectors 242a and 242b may be due to the distal facet reflection beam 232.

In a typical lithotripsy procedure, there are many time periods where the target 106 is far enough away from the distal end 126 of the optical fiber 104 (e.g., greater than or equal to 4 millimeters, or the like) such that signals reaching the signal detectors 242a and 242b are originating only from the distal end 126 of the optical fiber 104 (e.g., are only distal facet reflection beam 232).

The processing unit 112 can execute instructions 128 to identify periods where the distance 130 is above a threshold value (e.g., 4 mm, 6 mm, 8 mm, or the like) for a long enough time and can measure the distal facet reflection beam 232 at this time and update and/or calibrate the overall distance 130 measurement algorithm based on this measurement. Accordingly, the distal facet reflection beams 232 can be periodically updated and accounted for during the procedure, which provides a more accurate measurement of the distance 130 between the distal end 126 of the optical fiber 104 and the target 106.

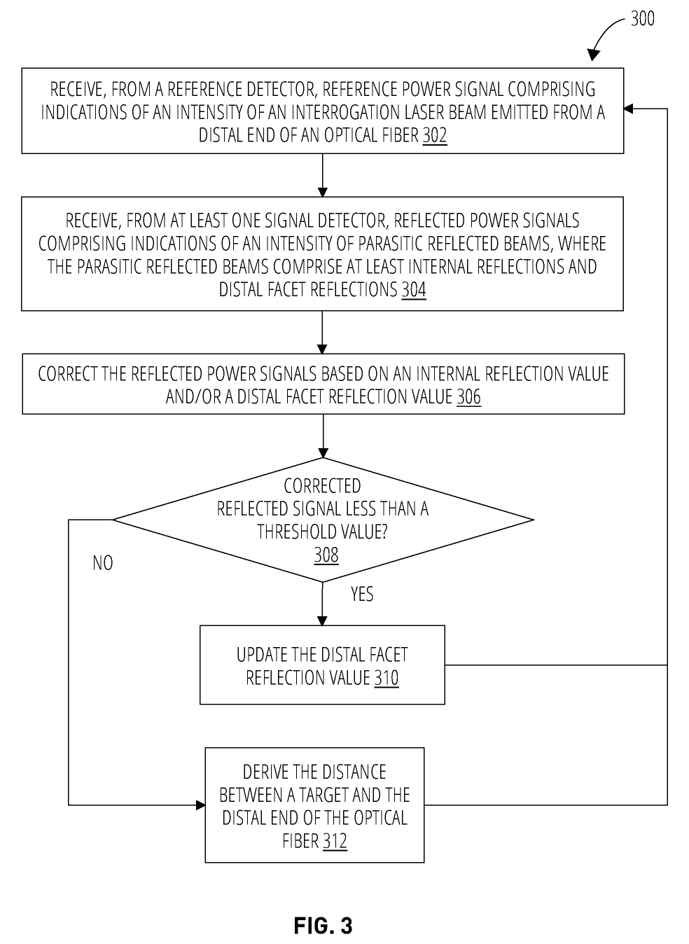

FIG. 3 illustrates a flowchart showing a method 300 for calibrating a distance measurement system. The method 300 is described with reference to the surgical laser system 100 of FIG. 1A and to the interrogation sub-system 200 of FIG. 2. It is to be appreciated however, that the method 300 could be implemented by a surgical system different than the one depicted herein.

The method 300 can begin at block 302. At block 302 “receive, from a reference detector, reference power signal comprising indications of an intensity of an interrogation laser beam emitted from a distal end of an optical fiber” a reference power signal comprising indications of an intensity of an interrogation laser beam emitted from a distal end of an optical fiber can be received. For example, processing unit 112 can execute instructions 128 to receive from reference detector 210, signals comprising indications of the power of interrogation laser beams 208. As noted above, interrogation laser beams 208 can comprise multiple components (e.g., laser beams 204a, 204b, 204c, etc.) In some examples, processing unit 112 can execute instructions 128 to receive indications of a power of each respective component.

Continuing to block 304 “receive, from at least one signal detector, reflected power signals comprising indications of an intensity of parasitic reflected beams, where the parasitic reflected beams comprise at least internal reflections and distal facet reflections” reflected power signals comprising indications of an intensity of parasitic reflected beams can be received, where the parasitic reflected beams comprise at least internal reflections and distal facet reflections. For example, processing unit 112 can execute instructions 128 to receive from signal detectors 242a and/or 242b, signals comprising indications of the power of parasitic reflected beams 238. As noted above, parasitic reflected beams 238 can comprise multiple components (e.g., distal facet reflection beam 232, proximal facet reflection beam 234, internal reflection beam 236, etc.) In some examples, proximal facet reflection beams 234 can be optically eliminated from the parasitic reflected beams 238 (e.g., by light snatch mirror 244 and light snatch detector 246, or the like). As such, signals generated by signal detectors 242a and 242b will depend only on distal facet reflection beam 232 and internal reflection beam 236. Further, as introduced above there will be periods of time during a procedure where the target 106 is far enough away from the distal end 126 of the optical fiber 104 that all of interrogation laser beams 208 will be absorbed by the liquid medium, and as such, target reflection beam 230 will be substantially zero, or will be small enough to allow calibration as described herein.

Continuing to block 306 “correct the reflected power signals based on an internal reflection value and/or a distal facet reflection value” the reflected power signals can be corrected based on an internal reflection value and a distal facet reflection value. As used herein, the internal reflection value is the known value of the contribution of the internal reflection beam 236; while the distal reflection value is the derived value of the contribution of the distal facet reflection beam 232, which is repeatedly updated as described herein.

With some embodiments, processing unit 112 can execute instructions 128 to derive the corrected reflected power signals as the difference between the reflected power signals and the internal reflection value and the distal facet reflection value. Said differently, processing unit 112 can execute instructions 128 to subtract the internal reflection value and the distal facet reflection value from each component (or selected components) of the reflected power signals.

In some embodiments, processing unit 112 can execute instructions 128 to derive the corrected reflected power signals as the difference between the reflected power signals and the internal reflection value only. Said differently, processing unit 112 can execute instructions 128 to subtract the internal reflection value from each component (or selected components) of the reflected power signals.

As noted, processing unit 112 can execute instructions 128 to derive corrected reflected power signals for select components. For example, described above, the interrogation laser beams 208 may have multiple components (e.g., laser beams 204a, 204b, and 204c). As a specific example, laser beams 204a, 204b, and 204c may have approximate wavelengths of 1310 nm, 1340 nm, and 1431 nm. In such an example, processing unit 112 can execute instructions 128 to derive corrected reflected power signals for the 1310 nm and 1340 nm components of the signals.

Continuing to decision block 308 “corrected reflected signals less than a threshold value?” a determination is made as to whether the corrected reflected signals are less than a threshold value. For example, processing unit 112 can execute instructions 128 to determine whether the corrected reflected signals are less than a threshold value, such as, a corrected signal threshold value. In some embodiments, the corrected signal threshold value can include a value for each component of the reflected power signals, or for the selected components for which corrected reflected power signals are derived. With some embodiments (e.g., where the corrected reflected power signal is based on both the internal reflection value and the distal facet reflection value) the corrected signal threshold values can be a predefined absolute threshold. With other examples (e.g., where the corrected reflected power signal is based on only the internal reflection value) the corrected signal threshold values can be based on the most recently derived distal facet reflection value). For example, in some embodiments, the threshold can be less than or equal to 5% of the prior distal facet reflection value, less than or equal to 3% of the prior distal facet reflection value, or less than or equal to 1.5% of the prior distal facet reflection value.

From decision block 308, method 300 can continue to block 310 or block 312. For example, where a determination is made that the corrected reflected signals are less than the threshold value, method 300 can continue from decision block 308 to block 310. In some embodiments, where a determination is made than any one component of the corrected reflected signals (e.g., 1310 nm component, 1340 nm component, or the like) is less than the threshold value then the method 300 can continue from decision block 308 to block 310.

Alternatively, where a determination is made that the corrected reflected signals are not less than the threshold value, method 300 can continue from decision block 308 to block 312. In some embodiments, only where a determination is made than all of the selected components of the corrected reflected signals (e.g., 1310 nm component and 1340 nm component, or the like) are not less than the threshold value will the method 300 continue from decision block 308 to block 312.

At block 310 “update the distal facet reflection value” the distal facet reflection value can be updated. For example, processing unit 112 can execute instructions 128 to derive an updated distal facet reflection value to calibrate the interrogation sub-system 200 as outlined herein. As a specific example, processing unit 112 can execute instructions 128 to update the distal facet reflection value based on the prior measured signal minus the internal reflection value for each component of the reflected signal (e.g., 1310 nm, 1340 nm, 1431 nm, or the like).

At block 312 “derive the distance between a target and the distal end of the optical fiber” a distance between the target and the distal end of the optical fiber can be derived. For example, processing unit 112 can execute instructions 128 to derive the distance 130 between the distal end 126 of the optical fiber 104 and the target 106. In general, the distal can be derived based on a ratio of the target reflection beam 230 and the interrogation laser beams 208. Said differently, processing unit 112 can execute instructions 128 to derive the distance 130 based on a ratio of the output from the reference detector 210 and the signal detectors 242a and 242b where the output from the signal detectors 242a and 242b are corrected based on the internal reflection value and the distal facet reflection value to remove contributions of distal facet reflection beam 232 and internal reflection beam 236 from the signals.

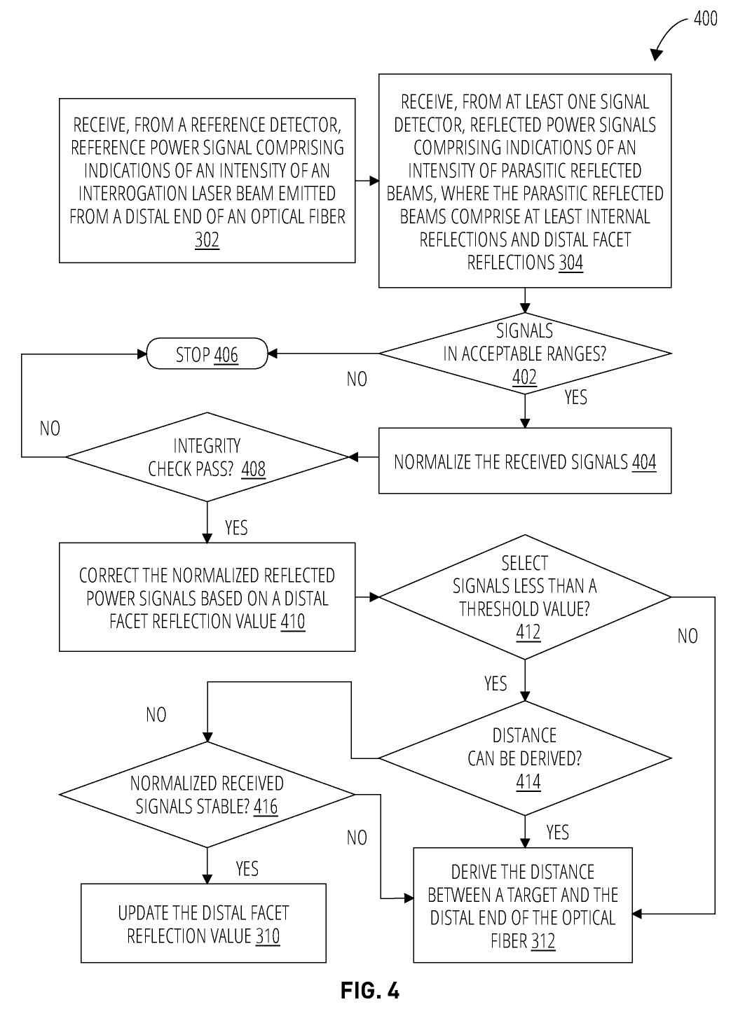

FIG. 4 illustrates a flowchart showing a method 400 for calibrating a distance measurement system. The method 400 is described with reference to the surgical laser system 100 of FIGS. 1A and 1B, to the interrogation sub-system 200 of FIG. 2, and to the method 300 of FIG. 3. It is to be appreciated however, that the method 400 could be implemented by a surgical system different than the one depicted herein. Further, method 400 includes several steps or operations similar to the steps of operations of method 300. As such, where appropriate, reference to steps or operations of method 300 are made for conciseness.

The method 400 can begin at block 302 and continue to block 304 and then to decision block 402. At decision block 402 “signals in acceptable ranges?” a determination can be made as to whether the signals are in acceptable ranges. For example, processing unit 112 can execute instructions 128 to determine whether the signals received at blocks block 302 and block 304 are within a raw signal threshold range of values. From decision block 402, method 400 can continue to block 404 or done block 406. For example, where a determination is made that the received signals are within the raw signal threshold range of values, the method 400 can continue from decision block 402 to block 404. Alternatively, where a determination is made that any one of the received signals are not within the raw signal threshold range of values, the method 400 can continue from decision block 402 to done block 406.

At done block 406, method 400 can terminate. In some embodiments, processing unit 112 can execute instructions 128 to generate an error and display the error on a display screen associated with the interrogation sub-system 200. At block 404 “normalize the received signals” the received signals (e.g., the signals received at block 302 and block 304) can be normalized to remove bias. With some embodiments, the signals can be smoothed prior to normalization. For example, processing unit 112 can execute instructions 128 to apply a smoothing filter to the received reference signals and the received reflected signals to reduce noise. Further, processing unit 112 can execute instructions 128 to normalize the received signals to remove contributions to each signal from dark current.

Continuing to decision block 408 “integrity check pass?” a determination can be made as to whether the integrity of the optical fiber 104 passes known check. For example, processing unit 112 can execute instructions 128 to determine whether a standard deviation of the N last samples of the normalized values is less than a fiber integrity threshold value. From decision block 408, method 400 can continue to block 410 or return to done block 406. For example, where a determination is made that the integrity check passed, the method 400 can continue from decision block 408 to block 410. Alternatively, where a determination is made that the integrity check did not pass, the method 400 can return to done block 406 from decision block 408.

At block 410 “correct the normalized reflected power signals based on a distal facet reflection value” corrected reflected power signals can be generated based on the normalized reflected power signals and a distal facet reflection value. As used herein, the distal reflection value is the derived value of the contribution of the distal facet reflection beam 232, which is repeatedly updated as described herein. In general, corrected reflected power signals are derived based on subtracting both the internal reflections and the last known fiber distal end reflection normalized signals from the normalized signals associated with components of the interrogation laser beams 208 having low water absorption coefficients (e.g., the 1310 nm and 1340 nm laser sources, or the like). As such, the resulted signals represent reflections only from the target (e.g., target reflection beam 230) and eliminates internal reflections (e.g., internal reflection beam 236) and reflections from the distal end 126 of the optical fiber 104 (e.g., distal facet reflection beam 232). It is noted that the subtraction is done twice, once without considering the normalized signals associated with the calibration component of the interrogation laser beams 208 (e.g., the 1431 nm laser source, or the like) and once with correcting for the current known distal facet reflection values for the high and low water absorption components of the interrogation laser beams 208.

Further, the correction is conducted using the ratio between the normalized signal of the calibration component signal (e.g., the 1431 nm laser signal) and its last known fiber distal end reflection normalized signals (e.g., the distal facet reflection value). This is to compensate for changes in the reflections from the distal end 126 (e.g., distal facet reflection beams 232) that are represented by the calibration component but not incorporated yet into the last known distal facet reflection values for the high and low absorption components.

Continuing to decision block 412 “select signals less than a threshold value?” a determination is made as to whether selected ones of the corrected reflected signals are less than a threshold value. For example, processing unit 112 can execute instructions 128 to determine whether the selected ones (e.g., the 1310 nm and 1340 nm component, or the like) of the corrected reflected signals are less than a threshold value, such as, corrected signal threshold values. In some embodiments, the corrected signal threshold values can include a value for each of the selected components of the reflected power signals.

From decision block 412, method 400 can continue to decision block 414 or block 312. For example, where a determination is made that the selected corrected reflected signals are less than the threshold value, method 400 can continue from decision block 412 to decision block 414. In some embodiments, where a determination is made than any one component of the selected corrected reflected signals (e.g., 1310 nm component, 1340 nm component, or the like) is less than the threshold value then the method 400 can continue from decision block 412 to decision block 414.

Alternatively, where a determination is made that the selected corrected reflected signals are not less than the threshold value, method 400 can continue from decision block 412 to block 312. In some embodiments, only where a determination is made than all of the selected components of the corrected reflected signals (e.g., 1310 nm component and 1340 nm component, or the like) are not less than the threshold value will the method 400 continue from decision block 412 to block 312.

At decision block 414 “distance can be derived?” a determination can be made as to whether the distance can be derived. For example, processing unit 112 can execute instructions 128 to determine whether the corrected reflected signals are within ranges suitable for deriving the distance 130 between the distal end 126 of the optical fiber 104 and the target 106. As a specific example, processing unit 112 can execute instructions 128 that the corrected reflected signals are positive and within a specified range and determine that the distance can be derived based on such a determination. With some embodiments, the specified range can correspond to a distance between 0 mm and 4 mm, between 0 mm and 6 mm, or between 0 mm and 8 mm. From decision block 414, method 400 can continue to block 312 or decision block 416. For example, where a determination is made that the distance can be derived, method 400 can continue from decision block 414 to block 312; while where a determination is made that the distance cannot be derived, the method 400 can continue from decision block 414 to decision block 416.

At decision block 416 “normalized received signals stable?” a determination can be made as to whether the normalized received signals are stable. For example, processing unit 112 can execute instructions 128 to determine whether the derived standard deviation of the normalized reflected power signals is less than a stability threshold value. From decision block 416, method 400 can continue to block 310 or block 312. For example, where a determination is made that the normalized received signals are stable, the method 400 can continue from decision block 416 to block 310; while where a determination is made that the normalized received signals are not stable, the method 400 can continue from decision block 416 to block 312.

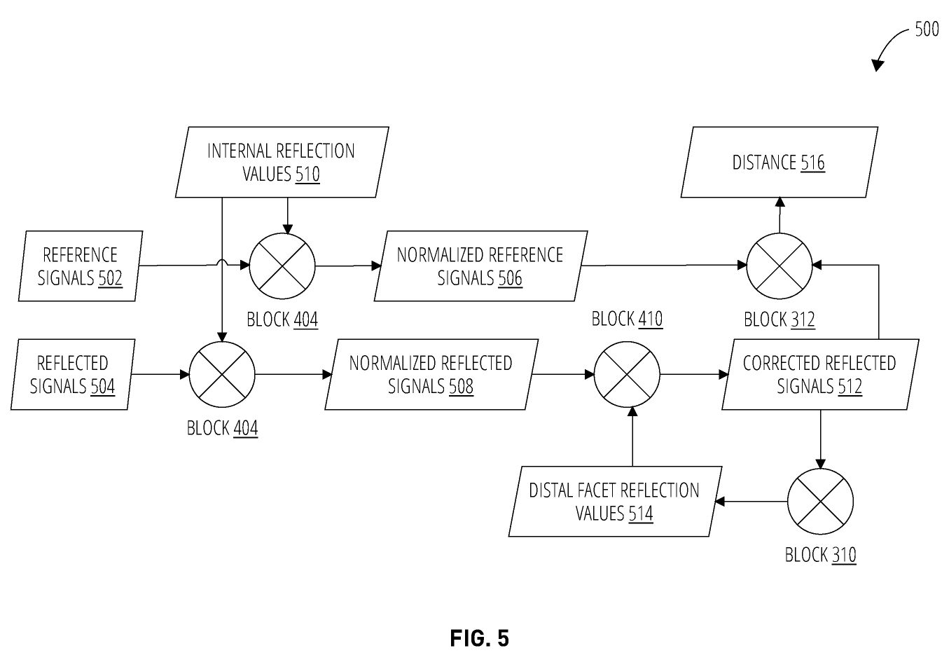

FIG. 5 illustrates an example dataflow diagram 500 showing signals and threshold values described herein as part of a distance measurement process and/or a calibration process for a distance measurement system. Dataflow diagram 500 refers to a number of data structures, which can be stored in memory 114 and accessed by processing unit 112 in executing instructions 128. Further, dataflow diagram 500 refers to the interrogation sub-system 200 of FIG. 2 as well as steps or operations of method 300 of FIG. 3 and method 400 of FIG. 4 for convenience in describing the diagram.

The dataflow diagram 500 can begin with reference signals 502 and reflected signals 504. In general, reference signals 502 can correspond to the signals for each component of interrogation laser beams 208 received from reference detector 210 while reflected signals 504 can correspond to signals for each component of interrogation laser beams 208 received from signal detectors 242a and 242b, which can correspond to target reflection beam 230, distal facet reflection beam 232, and/or internal reflection beam 236.

At block 404, normalized reference signals 506 and normalized reflected signals 508 are derived (e.g., by processing unit 112 executing instructions 128, or the like). For example, normalized reference signals 506 and normalized reflected signals 508 can be derived by subtracting internal reflection values 510 from reference signals 502 and reflected signals 504.

At block 410, corrected reflected signals 512 is derived (e.g., by processing unit 112 executing instructions 128, or the like). For example, corrected reflected signals 512 can be derived by subtracting distal facet reflection values 514 from normalized reflected signals 508. Given corrected reflected signals 512, either distal facet reflection values 514 can be updated or the distance 516 can be derived. For example, where the distance 130 between the distal end 126 of the optical fiber 104 and the target 106 is greater than a threshold (e.g., 4 mm, 6 mm, 8 mm, or the like) distal facet reflection values 514 can be updated based on an average of N prior corrected reflected signals 512. Alternatively, where the distance 130 between the distal end 126 of the optical fiber 104 and the target 106 is less than the threshold the distance 516 can be derived.

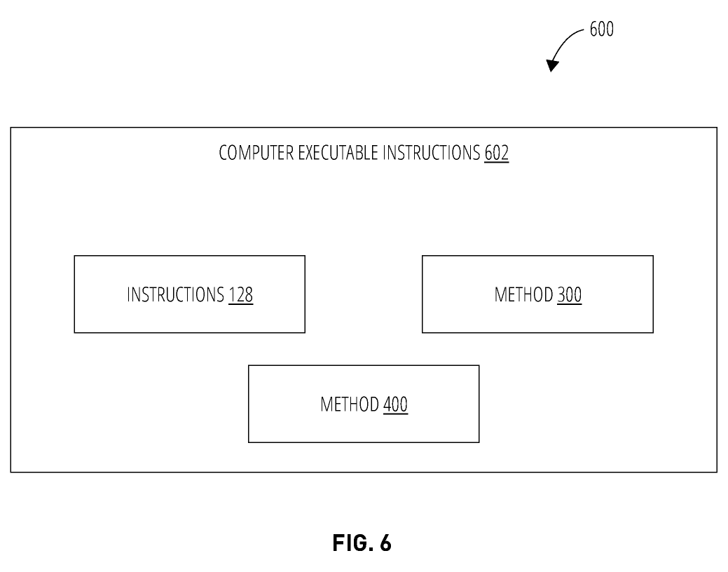

FIG. 6 illustrates computer-readable storage medium 600. Computer-readable storage medium 600 may comprise any non-transitory computer-readable storage medium or machine-readable storage medium, such as an optical, magnetic or semiconductor storage medium. In various embodiments, computer-readable storage medium 600 may comprise an article of manufacture. In some embodiments, computer-readable storage medium 600 may store computer executable instructions 602 with which circuitry (e.g., processing unit 112, or the like) of a surgical laser system or console (e.g., surgical laser console 102) can execute. For example, computer executable instructions 602 can include instructions to implement operations described with respect to instructions 128, method 300, and/or method 400. Examples of computer-readable storage medium 600 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 602 may include any suitable type of code, such as source code, compiled code, interpreted code, executable code, static code, dynamic code, object-oriented code, visual code, and the like.

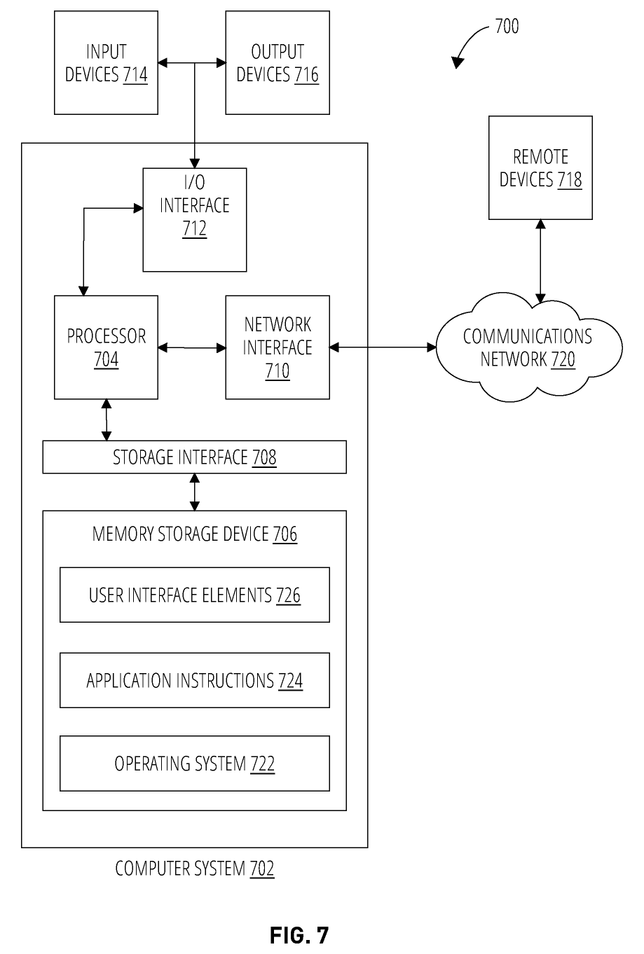

FIG. 7 is a block diagram of a computing environment 700 including a computer system 702 for implementing embodiments consistent with the present disclosure. In some embodiments, the computing environment 700, or portion thereof (e.g., the computer system 702) may comprise or be comprised in a surgical laser console (e.g., surgical laser console 102, or the like). Accordingly, in various embodiments, computer system 702 may be used to calibrate a process to measure a distance 130 between the distal end 126 of the optical fiber 104 and the target 106 to account for reflections from the facet (or surface) of the distal end 126 as outlined above.

The computer system 702 may include a central processing unit (“CPU” or “processor”) 704. The processor 704 may include at least one data processor for executing instructions and/or program components for executing user or system-generated processes. A user may include a person, a person using a device such as those included in this disclosure, or another device. The processor 704 may include specialized processing units such as integrated system (bus) controllers, memory management control units, floating point units, graphics processing units, neural processing units, digital signal processing units, etc. The processor 704 may be disposed in communication with input devices 714 and output devices 716 via I/O interface 712. The I/O interface 712 may employ communication protocols/methods such as, without limitation, audio, analog, digital, stereo, IEEE-1394, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), IEEE 802.n /b/g/n/x, Bluetooth, cellular (e.g., Code-Division Multiple Access (CDMA), High-Speed Packet Access (HSPA+), Global System For Mobile Communications (GSM), Long-Term Evolution (LTE), or the like), etc.

Using the I/O interface 712, computer system 702 may communicate with input devices 714 and output devices 716. In some embodiments, the processor 704 may be disposed in communication with a communications network 720 via a network interface 710. In various embodiments, the communications network 720 may be utilized to communicate with a remote memory storage device 706, such as for accessing look-up tables, performing updates, or utilizing external resources. The network interface 710 may communicate with the communications network 720. The network interface 710 may employ connection protocols including, without limitation, direct connect, Ethernet (e.g., twisted pair 10/100/1000 Base T), Transmission Control Protocol/Internet Protocol (TCP/IP), token ring, IEEE 802.11a/b/g/n/x, etc.

The communications network 720 can be implemented as one of the different types of networks, such as intranet or Local Area Network (LAN), Closed Area Network (CAN) and such. The communications network 826 may either be a dedicated network or a shared network, which represents an association of the different types of networks that use a variety of protocols, for example, Hypertext Transfer Protocol (HTTP), CAN Protocol, Transmission Control Protocol/Internet Protocol (TCP/IP), Wireless Application Protocol (WAP), etc., to communicate with each other. Further, the communications network 720 may include a variety of network devices, including routers, bridges, servers, computing devices, storage devices, etcetera. In some embodiments, the processor 704 may be disposed in communication with a memory storage device 706 via a storage interface 708. The storage interface 708 may connect to memory storage device 706 including, without limitation, memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etcetera.

Furthermore, memory storage device 706 may include one or more computer-readable storage media utilized in implementing embodiments consistent with the present disclosure. Generally, a computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, non-volatile memory, hard drives, Compact Disc (CD) ROMs, Digital Video Disc (DVDs), flash drives, disks, and any other known physical storage media.

The memory storage device 706 may store a collection of program or database components, including, without limitation, an operating system 722, application instructions 724, and user interface elements 726. In various embodiments, the operating system 722 may facilitate resource management and operation of the computer system 702. Examples of operating systems include, without limitation, APPLE^{®} MACINTOSH^{®} OS X^{®}, UNIX^{®}, UNIX-like system distributions (E.G., BERKELEY SOFTWARE DISTRIBUTION^{®} (BSD), FREEBSD^{®}, NETBSD^{®}, OPENBSD^{®}, etc.), LINUX^{®} DISTRIBUTIONS (E.G., RED HAT^{®}, UBUNTU^{®}, KUBUNTU^{®}, etc.), IBM^{®}OS/2^{®}, MICROSOFT^{®} WINDOWS^{®} (XP^{®}, VISTA^{®}/7/8, 10 etc.), APPLE^{®} IOS^{®}, GOOGLE^{TM} ANDROID^{TM}, BLACKBERRY^{®} OS, or the like.

The application instructions 724 may include instructions that when executed by the processor 704 cause the processor 704 to perform one or more techniques, steps, procedures, and/or methods described herein, such to irrigate a site and irradiate a site as outlined herein. For example, application instructions 724, when executed by processor 704 can cause processor 704 to perform the method 300, and/or method 400.

The user interface elements 726 may facilitate display, execution, interaction, manipulation, or operation of program components through textual or graphical facilities. For example, user interfaces may provide computer interaction interface elements on a display system operatively connected to the computer system 702, such as cursors, icons, checkboxes, menus, scrollers, windows, widgets, etcetera. The user interface elements 726 may be employed by application instructions 724 and/or operating system 722 to provide, for example, a user interface with which a user can interact with computer system 702. As a specific example, the distance 516 can be displayed on a display. In some embodiments, the user interface elements 726 may be integrated with the display (not shown).

Terms used herein should be accorded their ordinary meaning in the relevant arts, or the meaning indicated by their use in context, but if an express definition is provided, that meaning controls.

Herein, references to "one embodiment" or "an embodiment" do not necessarily refer to the same embodiment, although they may. Unless the context clearly requires otherwise, throughout the description and the claims, the words "comprise," "comprising," and the like are to be construed in an inclusive sense as opposed to an exclusive or exhaustive sense; that is to say, in the sense of "including, but not limited to." Words using the singular or plural number also include the plural or singular number respectively, unless expressly limited to a single one or multiple ones. Additionally, the words "herein," "above," "below" and words of similar import, when used in this application, refer to this application as a whole and not to any particular portions of this application. When the claims use the word "or" in reference to a list of two or more items, that word covers all of the following interpretations of the word: any of the items in the list, all of the items in the list and any combination of the items in the list, unless expressly limited to one or the other. Any terms not expressly defined herein have their conventional meaning as commonly understood by those having skill in the relevant art(s).

What is claimed is:

1. A surgical laser system, comprising:
at least one laser source configured to output an interrogation laser beam;
at least one signal detector configured to measure an intensity of a reflected light beam;
a processor; and
a memory comprising instructions, which when executed by the processor cause the processor to:
send a control signal to the at least one laser source to cause the at least one laser source to generate the interrogation laser beam,
receive electrical signals from the at least one signal detector, the electrical signals comprising indications of an intensity of a reflected light beam, wherein the reflected light beam comprises target reflections and parasitic reflections, and wherein the reflected light beam is responsive to incidence of the interrogation laser beam on a target,
derive a corrected intensity based on the intensity and one or more parasitic intensities,
determine whether the corrected intensity is less than a threshold value, and
update at least one of the parasitic intensities based on the corrected intensity responsive to a determination that the corrected intensity is less than the threshold value.

2. The surgical laser system of claim 1, the instructions when executed by the processor further cause the processor to derive the normalized intensity based on the measured intensity and a dark current intensity and to derive the corrected intensity is based on the normalized intensity and the one or more parasitic intensities.

3. The surgical laser system of claim 2, the instructions when executed by the processor further cause the processor to apply a smoothing filter to the measured intensity prior to deriving the normalized intensity.

4. The surgical laser system of claim 3, the instructions when executed by the processor further cause the processor to receive electrical signals from a reference detector, the electrical signals comprising indications of an intensity of the interrogation laser beam and to derive the normalized intensity based on the intensity of the reflected light beam, the intensity of the interrogation laser beam, and the dark current intensity.

5. The surgical laser system of claim 4, the instructions when executed by the processor further cause the processor to:
determine whether a standard deviation of the normalized intensity value for N last measured intensities is less than a stability threshold value; and
update the at least one of the parasitic intensities based on the corrected intensity responsive to the determination that the corrected intensity is less than the threshold value and a determination that the standard deviation of the normalized intensity value for the N last measured intensities is less than the stability threshold value.

6. The surgical laser system of claim 5, the instructions when executed by the processor further cause the processor to receive electrical signals from the at least one signal detector during a period when the interrogation laser beam is off, the electrical signals comprising indications of the dark current intensity.

7. The surgical laser system of claim 1, wherein the one or more parasitic intensities comprise a distal facet reflection value and an internal reflection value.

8. The surgical laser system of claim 7, the instructions when executed by the processor further cause the processor to subtract the distal facet reflection value and the internal reflection value from the measured intensity to derive the corrected intensity when the threshold value is substantially zero (0).

9. The surgical laser system of claim 8, the instructions when executed by the processor further cause the processor to set the distal facet reflection value as the corrected intensity minus the internal reflection value to update at least one of the parasitic intensities based on the corrected intensity.

10. The surgical laser system of claim 7, the instructions when executed by the processor further cause the processor to subtract the internal reflection value from the measured intensity to derive the corrected intensity when the threshold value is equal to the distal facet reflection value.

11. The surgical laser system of claim 10, the instructions when executed by the processor further cause the processor to set the distal facet reflection value as the corrected intensity to update at least one of the parasitic intensities based on the corrected intensity.

12. The surgical laser system of claim 1, wherein the interrogation laser beam comprises a plurality of wavelength components, wherein the electrical signals received from the at least one signal detector comprises indications of the intensity of the reflected light beam at each of the plurality of wavelength components, wherein the one or more parasitic intensities comprises one or more parasitic intensities at each of the plurality of wavelength components, wherein the corrected intensity comprises a corrected intensity at each of the plurality of wavelength components, wherein the threshold value comprises a threshold value at each of the plurality of wavelength components, and wherein the instructions when executed by the processor further cause the processor to:
determine whether the corrected intensity is less than the threshold value comprises determining, for each of the plurality of wavelength components, whether the corrected intensity is less than the respective threshold value, and
updating, for each of the wavelength components, at least one of the parasitic intensities based on the corrected intensity responsive to a determination that the corrected intensity is less than the respective threshold value.

13. The surgical laser system of claim 12, wherein the first wavelength component is substantially 1310 nanometers (nm), wherein the second wavelength component is substantially 1340 nm, and wherein the third wavelength component is substantially 1431 nm.

14. The surgical laser system of claim 1, the instructions when executed by the processor further cause the processor to estimate a distance between a distal end of the optical fiber and the target based on the corrected intensity responsive to a determination that the corrected intensity is not less than the threshold value.

15. At least one machine readable storage device comprising instructions that when executed by a processor of a surgical laser system cause the surgical system to:
generate, via a laser source, an interrogation laser beam;
illuminate, via an optical fiber coupled to the surgical laser system, a target with the interrogation laser beam;
measure, via at least one signal detector, an intensity of a reflected light beam, wherein the reflected light beam comprises target reflections and parasitic reflections;
derive a corrected intensity based on the measured intensity and one or more parasitic intensities;
determine whether the corrected intensity is less than a threshold value; and
update at least one of the parasitic intensities based on the corrected intensity responsive to a determination that the corrected intensity is less than the threshold value.

16. The at least one machine readable storage device of claim 15, the instructions when executed by the processor further cause the surgical laser system to:
measure, via the at least one signal detector, a dark current intensity during a period when the interrogation laser beam is off; and
derive a normalized intensity based on the measured intensity and the dark current intensity,
wherein deriving the corrected intensity is based on the normalized intensity and the one or more parasitic intensities.

17. The at least one machine readable storage device of claim 15,
wherein the interrogation laser beam comprises a plurality of wavelength components,
wherein measuring, via the at least one signal detector, the intensity of the reflected light beam comprises measuring the intensity of the reflected light beam at each of the plurality of wavelength components,
wherein the one or more parasitic intensities comprises one or more parasitic intensities at each of the plurality of wavelength components,
wherein deriving the corrected intensity based on the measured intensity and the one or more parasitic intensities comprises deriving, for each of the plurality of wavelength components, the corrected intensity based on the measured intensity and the one or more parasitic intensities at the respective wavelength component,
wherein the threshold value comprises a threshold value at each of the plurality of wavelength components,
wherein determining whether the corrected intensity is less than the threshold value comprises determining, for each of the measured intensities, whether the measured intensity is less than the threshold value at the respective wavelength component, and
wherein updating at least one of the parasitic intensities based on the corrected intensity responsive to a determination that the corrected intensity is less than the threshold value comprises updating, for each of the wavelength components, at least one of the parasitic intensities at the respective wavelength component.

18. A method for a surgical laser system, comprising:
generating, via a laser source, an interrogation laser beam;
illuminating, via an optical fiber coupled to the surgical laser system, a target with the interrogation laser beam;
measuring, via at least one signal detector, an intensity of a reflected light beam, wherein the reflected light beam comprises target reflections and parasitic reflections;
deriving a corrected intensity based on the measured intensity and one or more parasitic intensities;
determining whether the corrected intensity is less than a threshold value; and
updating at least one of the parasitic intensities based on the corrected intensity responsive to a determination that the corrected intensity is less than the threshold value.

19. The method of claim 18, further comprising:
measuring, via the at least one signal detector, a dark current intensity during a period when the interrogation laser beam is off; and
deriving a normalized intensity based on the measured intensity and the dark current intensity,
wherein deriving the corrected intensity is based on the normalized intensity and the one or more parasitic intensities.

20. The method of claim 18,
wherein the interrogation laser beam comprises a plurality of wavelength components,
wherein measuring, via the at least one signal detector, the intensity of the reflected light beam comprises measuring the intensity of the reflected light beam at each of the plurality of wavelength components,
wherein the one or more parasitic intensities comprises one or more parasitic intensities at each of the plurality of wavelength components,
wherein deriving the corrected intensity based on the measured intensity and the one or more parasitic intensities comprises deriving, for each of the plurality of wavelength components, the corrected intensity based on the measured intensity and the one or more parasitic intensities at the respective wavelength component,
wherein the threshold value comprises a threshold value at each of the plurality of wavelength components,
wherein determining whether the corrected intensity is less than the threshold value comprises determining, for each of the measured intensities, whether the measured intensity is less than the threshold value at the respective wavelength component, and
wherein updating at least one of the parasitic intensities based on the corrected intensity responsive to a determination that the corrected intensity is less than the threshold value comprises updating, for each of the wavelength components, at least one of the parasitic intensities at the respective wavelength component.
